(19) United States
(12) Patent Application Publication
(10) Pub. No.: US 2010/0015143 A1
(43) Pub. Date: Jan. 21, 2010

(54) Compositions and Methods Relating to Modulation of Immune System Components

(76) Inventors: Tracy Hussell, Kent (GB); James W. Larrick, Sunnyvale, CA (US); George L. Foltin, New York, NY (US)

Correspondence Address: David M. McConoughey, 179 Indiana St., Maplewood, NJ, 07040, US

(21) Appl. No.: 12/225,459

(22) PCT Filed: Mar. 22, 2007
(86) PCT No.: PCT/US2007/007098

§ 371 Date: Apr. 24, 2009

Related U.S. Application Data
(60) Provisional application No. 60/785,407, filed on Mar. 22, 2006.

(51) Int. Cl. A61K 39/395; A61K 38/16

(52) U.S. Cl. 424/134.1; 514/12

(57) ABSTRACT

A composition comprising a molecular blockade agent to a costimulatory molecule which costimulatory molecule satisfies the following criteria: a. absent in naÊve or resting T-lymphocytes; b. inducible; c. expressed; and d. prominent at the height of an immunopathological response, such as a disease/condition response. Preferably, the costimulatory molecule is OX40 and the molecular blockade agent is an antibody or antibody fragment having antibody activity to OX40. Further, the system may involve modulation of the molecular signal pathway of the aforesaid costimulatory molecule.

### CLAIM OF PRIORITY

The benefit of the priority of earlier filed U.S. Patent Application Ser. No. 60/765,407, filed Mar. 22, 2007 is hereby claimed.

### TECHNICAL FIELD

The present invention relates to the production and regulation of molecules attendant upon an immune response in a biological system

### BACKGROUND

To give the present invention a context it should be considered that, for example, respiratory tract infections are responsible for a significant portion of all deaths from communicable diseases.

In general, the severity of disease is attributed to both the nature of the infection and the magnitude of the host immune response. The present invention is intended to address the latter causative factor, in particular the inappropriate or immunopathological response of the host's immune system to infection or to trauma.

### DESCRIPTION OF VARIOUS EMBODIMENTS OF THE INVENTION

### 1—Introduction

The present invention relates to a composition incorporating a molecular blockade agent to a costimulatory molecule, said costimulatory satisfying the following criteria:

a. absent in naïve or resting T-lymphocytes;
b. inducible;
c. expressed; and
d. prominent at the height of an immunopathological response, such as a disease and/or condition response.

In addition, the present invention relates to a method in which such a molecular blockade agent is administered to a subject, such as a mammal, and preferably a human subject, prior to or contemporaneously with the height of an immunopathological response.

The molecular blockade agent may be an antibody to said costimulatory molecule or a fragment thereof, said fragment having antibody activity to said costimulatory molecule. The costimulatory molecule may be a cytokine receptor or a correlative ligand to said receptor. While not a cytokine (being a transmembrane protein on T cells) OX40 is an example of a costimulatory molecule satisfying the foregoing criteria. And OX40L is also a candidate costimulatory molecule.

Examples of other candidate costimulatory TNFR family members are 4-1BB, CD27, HVEM, GITRR, CD30, as well as others as may be mentioned later. An example of a correlative ligand is 4-1BBL. Examples of additional-costimulatory molecules are ICOS, PD1, and CTLA4. Examples of candidate correlative ligands are CD70 [for CD30], LIGHT [for HVEM], GITRL, CD30L, as well as others as may be mentioned later.

Further, the present invention more broadly involves modulation of the molecular signal pathway of the aforesaid costimulatory molecule and its respective receptor or correlative ligand. An example of such a pathway is the TRAF 2 pathway of OX40 or 4-1BB. This modulation may include one or more of the pathway's extracellular components, transmembrane components, or intracellular components or a combination of two or more the foregoing. It should be borne in mind that components of a signaling pathway may be shared with other pathways and that a blockade may affect those other pathways.

Such compositions and methods may have use as research product, diagnostic, prophylactic, and therapeutic compositions for veterinary and human clinical use and as research, diagnostic, prophylactic, and therapeutic methods for veterinary and human clinical use, as well as composition selection, identification, and characterization methods. Such systems may have application against acute indications (as well as chronic), such as the immunopathology referred to as “cytokine storm” which may be occasioned by an influenza infection, such as pandemic influenza, for example Avian Influenza A (H5N1).

Illness to respiratory infection is mediated in part by T lymphocytes (“T cells”). It is not currently known whether the excessive T cells seen in the lung are due to recruitment, maintenance or both. Understanding how T cells are regulated during inflammation will therefore highlight novel avenues for intervention.

A molecular blockade agent may be made using standard well known methods making antibodies, antibody fragments having specified antibody activity, and agents having immunological activity against an antigen. Antibodies are generated to recognize foreign entities, such as foreign particles, (antigens). One method of making a molecular blockade antibody by screening a library of antibodies, finding those antibodies that react with the target costimulatory molecule such as OX40, and purifying it or them. Then an antibody fragment can be formed by cleaving off a portion of the antibody not required and PEGylating it. In the instance of OX40 this antibody fragment binds specifically to OX40, the costimulatory molecule, and the antibody fragment thereby blocks the ability of the OX40 (costimulatory molecule) to bind to OX40 ligand. As a result, the positive signal usually delivered to the T cells (by OX40 ligand) is blocked.

As used in the context of the present invention, the following terms are intended to comprehend the following associated meanings:

1. “absent”—not present;

2. “naïve”—not encountered and immunologically responded to an antigen before;

3. “resting”—possibly having previously encountered and immunologically responded to an antigen before, but not immunologically responding to an antigen presently;

4. “inducible”—not constitutively present, but capable of being up regulated or down regulated;

5. “expressed”—present;

6. “prominent”—a high level of expression on individual cells, as measured by comparing the levels of expression over a time course by flow cytometry and/or PCR, and preferably a level of at least 5%.

7. “costimulatory”—molecules that provide a stimulatory signal to T cells beyond that provided by simple recognition of the antigen. Co-stimulatory signals are required for full physiological activation of the T cells and are provided by membrane bound molecules on antigen presenting cells. Without this co-stimulatory signal the T cells are not fully activated and may even be permanently switched off.

8. “molecular blockade agent”—a reagent having blocking activity to a costimulatory molecule having the foregoing characteristics.

### 1.1 T Cells

T cells can be divided into two populations, T helper cells and T cytotoxic cells, according to their expression of the membrane bound glycoproteins CD4 and CD8, respectively. Cytotoxic T cells lyse infected or tumour cells after recognition of MHC class 1 molecules bearing foreign peptide, whereas CD4+ T cells bind MHC class II: peptide complexes and assist the cell expressing them. T helper cells can be further divided into three populations: Th1, Th2 and T regulatory cells. These subsets are defined according to the cytokines they produce —IFN-γ, TNF-α and IL-2 from Th1 cells; IL-4, IL-5 and IL-6 from Th2 cells; and IL-10 and TGF-β from T regs although IL-10 is also produced by Th2 cells. It should be noted, however, that T regs cannot be identified on the basis of their cytokine production alone. The cytokine profiles of these cell types allow them to induce discrete immune responses according to the nature of the threat. Th1 cytokines enable a cell-mediated immune response to target intracellular pathogens, whereas the Th2 response induces a humoral response targeting extracellular pathogens. T regulatory cells are able to suppress both of these responses, whereas Th1 and Th2 cells can only inhibit each other. Some studies imply that CD8+ T cells can also be subdivided on cytokine secretion profiles.

### 1.2 T Cell Co-Stimulation

For initial T cell activation at least two signals are required. The first, or primary, signal is transmitted when the T cell receptor binds to the self-MHC molecule bearing antigenic peptide on the antigen-presenting cell (APC). If only this signal is received, however, the T cell enters a state of anergy and becomes tolerant. In order that the cell passes into a fully activated state, a second, or secondary, signal, known as the co-stimulation signal, is necessary.

The most studied T cell co-stimulatory molecule is CD28, a type 1 transmembrane glycoprotein and a member of the Immunoglobulin superfamily. Engagement of CD28 with CD80 and CD86 on the APC enhances the T cell response by increasing IL-2 production, an autocrine T cell growth factor, and inducing the expression of Bcl-2, an anti-apoptotic gene. CD28 ligation also results in the rearrangement of the T cell plasma membrane and formation of the immunological synapse.

In addition to CD28, which remains the paradigm for co-stimulation, there are several other families of molecules, which facilitate subsequent T cell survival through successive rounds of division. Inducible co-stimulator (ICOS) is structurally related to CD28 but is not constitutively expressed on T cells. Rather, it is induced after activation on both CD4+ and CD8+ T cells. ICOS is expressed early following TCR-MHC interaction, peaking after 12-24 hours. Ligation of ICOS induces further T cell proliferation and may play a role in determining the cytokines produced. ICOS ligation does not lead to an increase in IL-2 production but rather IL-4, IL-5, IL-10, IFN-γ and TNF-α, indicating a role in determining the effector T cell phenotype.

The Tumour Necrosis Factor receptor superfamily is also involved in co-stimulation of T cells. This family includes OX40 (CD134) and 4-1 BB (CD137) as well as CD27 and HVEM. All are type 1 transmembrane proteins with several extracellular cysteine-rich domains.

### 1.3 OX40

OX40 (CD134) has a molecular weight of 47-50 KDa, with both O- and N-linked glycosylation. It contains an extracellular domain of 191 residues, a transmembrane region of 25 residues, and an intracellular tail of 36 residues. The extracellular domain contains three cysteine-rich domains, CRDs.

Both OX40 and 4-1BB are inducibly expressed 48-72 hours following T cell activation. Signaling through OX40 activates NF-κB through the TNF receptor associated factors TRAF-2 and -5. These bind to and activate NF-κB—inducing kinase (NIK), which in turn activates CHUK. CHUK is able to phosphorylated IκBα, which degrades, removing suppression from NF-κB and allowing it to translocate into the nucleus.

The co-stimulatory signal imparted by OX40 and 4-1BB ligation is important during late T cell proliferation and expansion; OX40-deficient mice show unaltered early T cell proliferation but enhanced apoptosis and reduced proliferation of T cells 4-5 days after TCR ligation. In addition, fewer memory cells develop. OX40 is expressed on CD4 and CD8 T cells, as well as B cells and dendritic cells. During inflammatory disease, OX40 is expressed on T cells at the sites of inflammation, including the lung, arthritic joint, and central nervous system.

The ligands to these receptors, OX40L and 4-1BBL, are also inducibly expressed by Toll-like receptor ligands and ligation of CD40 by T cells expressing CD40L, with kinetics of expression following the same pattern as that of their receptors on the T cell. Both molecules are type II transmembrane proteins that share homology with TNF and are expressed on B cells, macrophages and dendritic cells following activation. Although the interaction between the TNFRs and their ligands is known to be bi-directional, the nature of the benefit to the APC is, as yet, unknown. Since T cells play a pivotal role in immunopathology induced by infection, manipulation of late T cell co-stimulatory signals may represent a novel immune therapeutic strategy and correlative diagnostic and prophylactic strategies. The following is a summary of the co-stimulatory molecules on T cells and their function:

| Molecule on T cell | Family | Ligand on APC | Outcome of interaction |
|---|---|---|---|
| CD28 | CD28 | B7.1, B7.2 | Expansion of T cells in |
| | superfamily | (CD80, CD86) | primary infections, |
| | | | production of IL-2 |
| ICOS | CD28 | ICOSL | Production of cytokines |
| | superfamily | | including IL-4, IL-5, IL-10 |
| | | | and IFN-γ (not IL-2). |
| | | | Enhanced T-cell dependent |
| | | | B-cell help. |
| CTLA-4 | CD28 | B7.1, B7.2 | Decreased T cell activation |
| | superfamily | | and IL-2 synthesis, inhibition |
| | | | of CD28-mediated signal |
| | | | transduction. |
| PD-1 | CD28 | PD-L1, L2 | Inhibition of proliferation, |
| | superfamily | | inhibition of IFN- γ, IL-10 |
| | | | and IL-2 production. |
| HVEM/LIGHT | TNFR | LIGHT/HVEM | Co-stimulation, T-T |
| | | | interactions, proliferation of |
| | | | T cells, NF-κB activation, |
| | | | cytokine production, |
| | | | maturation of DCs |
| OX40 | TNFR | OX40L | Sustained CD4 T cell |
| | | | survival, memory |
| | | | development, proliferation of |
| | | | T cells. Greater effect on |
| | | | CD4 T cells |
| 4-1BB | TNFR | 4-1BBL | Sustained T cell survival, |
| | | | memory development. |
| | | | Greater effect on CD8 T |
| | | | cells |
| CD40L | TNF ligand | CD40 | Up-regulation of other co- |
| | family | | stimulatory molecules on |
| | | | APCs |
| CD27 | TNFR | CD70 | Expansion and proliferation |
| | | | of both CD4 and CD8 T |
| | | | cells, survival of effector T |
| | | | cells. Greater effect on |
| | | | secondary responses than |
| | | | primary |

### 1.3 Respiratory Infections and Immunopathology

Respiratory tract infections were responsible for 21.5% of all deaths from communicable diseases in 2001, according to the World Health Organisation, and new threats such as SARS and avian influenza are emerging continuously. The same study indicates that 98% of those deaths are due to lower respiratory tract infections, which can lead to pneumonia and bronchiolitis. The severity of disease is attributed to both the nature of the infection and the magnitude of the host immune response.

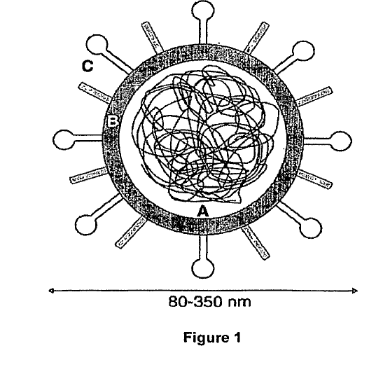

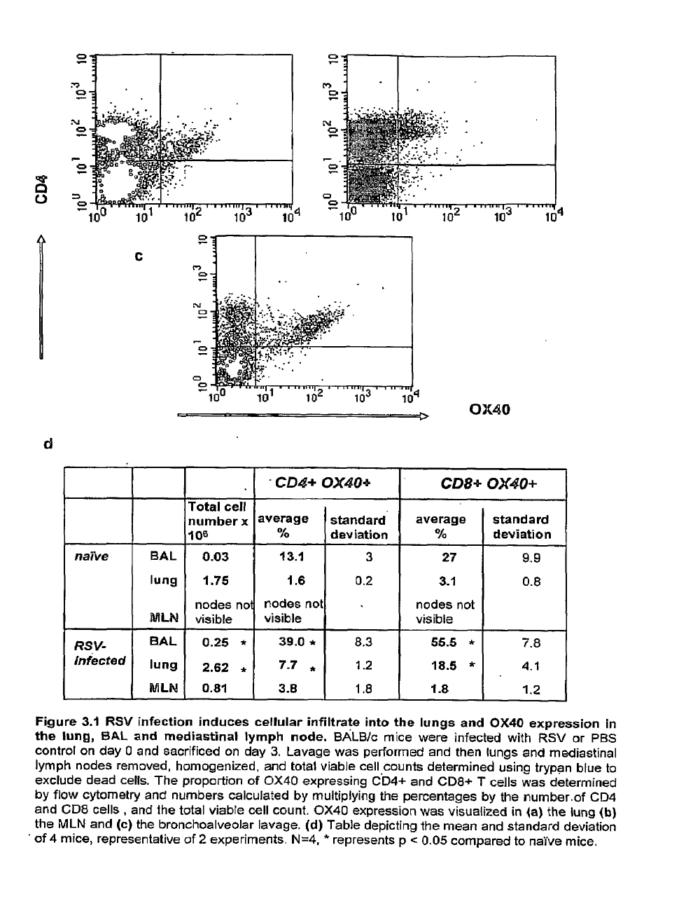

Respiratory syncytial virus (RSV) is the dominant cause of infant lower respiratory tract infection worldwide, responsible for 50% of infant bronchiolitis, with an infection rate of 70% in children below one year of age. Up to 4% of children infected with RSV require hospitalisation, and mortality rates exceed 70% in immune-compromised patients. RSV is from the Pneumovirus genus, Paramyxoviridae family, with single-stranded negative sense RNA encoding ten genes. RSV replicates in the nasopharynx after which it infects the respiratory epithelium through interaction of GAGs, and other unidentified receptors, on the cell surface with the RSV G and F surface glycoproteins. FIG. 1 is a schematic of the respiratory syncytial virus showing A—matrix, which contains the proteins M and M2; B—the capsid which is made up of a nucleoprotein and a phosphoprotein as well as the polymerase; and C—transmembrane proteins which include the fusion and attachment proteins. (FIG. 1 is taken from Medscape.com—Newborn Infant Nursing Reviews 2005.)

During infection the damage to the host and the symptoms displayed can be direct or indirect. Direct damage to the host depends on whether the virus is cytopathic (i.e. causes necrosis of the cell). Unlike influenza, RSV is a non-cytopathic virus and can establish a persistent infection in the host despite initial control by T cells. Bronchiolitis suffered during RSV infection is mainly caused by the large influx of host CD4+ and CD8+ T cells, macrophages, plasma cells and neutrophils into the airways. This leads to increased production of inflammatory cytokines, occlusion of the airways and reduced oxygen transfer. Previous attempts to develop a vaccine against RSV in the 1950s failed as immune memory to the vaccine heightened bronchiolitis during subsequent natural infection. In addition, since RSV infection itself does not induce sufficient memory to prevent re-infection in adults, it is perhaps not realistic to expect a formalin-inactivated vaccine strain to do so. To this end, we focus on reducing the numbers of Th1 CD4+ and CD8+ T cells which enter the airways during infection, thus reducing the production of inflammatory cytokines and damage to the epithelial cells of the airways. We therefore hypothesise that inhibiting late T cell co-stimulation will reduce the magnitude of the adaptive immune response, reducing occlusion, whilst leaving the resting naïve and memory T cell pools intact. In addition to testing this hypothesis during virus induced inflammation, this strategy may also be efficacious against autoimmune inflammatory disorders.

Previous work focussed on inhibiting OX40 by using a soluble fusion protein, OX40:Ig, during influenza infection where immunopathology causes occlusion of the airways. Blockade of OX40 reduces cachexia and weight loss without compromising viral clearance. Both CD4+ and CD8+ T cells are reduced, likely due to reduced proliferation, enhanced apoptosis and possibly reduced migration (OX40L is expressed on the inflamed endothelium). Stimulation through OX40 has also been tested during Cryptococcus neoformans infection through the use of an OX40L:Ig fusion protein. Unlike influenza, the disease caused by C. neoformans infection is attributed to enhanced pathogen replication due to limited T cell activation. The opposite strategy to influenza virus infection was therefore required. OX40 ligation on activated T cells increases IFN-γ production and reduces pulmonary eosinophilia. C. neoformans burden in the lung is also reduced.

### EXAMPLES

### Example 1

### Respiratory Syncytial Virus

### 2—Materials and Methods

### 2.1 Mice And Cell Lines.

8-12 week old female BALB/c and 9-10 week old male DBA/1 mice (Harlan Olac Ltd, Bicester, UK) were kept in pathogen free conditions according to Home Office guidelines. DO11.10 mice were bred in-house in animal facilities according to Home Office guidelines.

Bone marrow derived macrophages and dendritic cells were grown through removal of femurs from BALB/c mice, washing of the femur with RPMI, and plating with 2 μl MCSF or GM-CSF to 10 ml R10F (RPMI, 10% foetal calf serum, 2 nM/ml L-glutamine, 50 U/ml penicillin, 50 mg/ml streptomycin) containing 25 μM 2-mercaptoethanol. Medium was replaced after three days. DO11.10 splenocytes were removed from 6-10 week old mice and strained through a 100 μM sieve before red blood cell lysis was performed and the cells were incubated in RPMI with 10% FCS. The RAW 264.7 macrophage cell line was cultured in DMEM, 10% FCS, 50 U/ml penicillin, 50 mg/ml streptomycin, and split 1:3 every three days when confluent. For in vitro assays, 2×10⁶ cells were plated in 2 ml medium in each well of a 6 well plate and left for two hours to adhere before being treated with 100 ng/ml IFN-γ with or without 50 μg/ml OX40:1 g.

### 2.1a Purification of Cells.

CD4 cells were purified from single cell suspensions from DO11.10 spleens. Cells were resuspended at 10⁸ cells/ml in PBS containing 0.5% BSA and 2 mM EDTA, and 10% CD4 microbeads (Miltenyi Biotec) added. Cells were incubated for 15 minutes at 4° C. Cells were washed and resuspended in buffer and up to 10⁸ cells applied to one MS column in the presence of a magnetic field. Unlabelled cells were washed through with buffer and then the fraction containing the magnetically labelled cells was flushed out with a plunger. Cells were recounted and purity assessed by FACS.

### 2.2 OX40 Blocking Reagents.

The molecular blockade agent used was an OX40 blocking antibody reagent (“A9” obtained from Celltech R&D Limited, Slough, United Kingdom) which is a pegylated antibody fragment. A9 is a human IgG1 Fab fragment linked to polyethylene glycol, and is 40 KDa.

The murine OX40: mIgG1 fusion protein, OX40: Ig, and OX40L: mIgG1, OX40L: Ig, were obtained from Xenova Research Ltd (Cambridge, UK) and were constructed using a chimeric cDNA that contained the extracellular domain of either OX40 or OX40L fused to the constant region of murine IgG1. These constructs were used to transfect clonal Chinese hamster ovary cells and fusion proteins were purified from the culture supernatant using protein G sepharose (Taylor and Schwarz, j immunol methods 255:67-72).

### 2.3 Respiratory Syncytial Virus (RSV)

RSV (A2 strain) was grown on HEp-2 cell monolayers. RSV (1 pfu/cell) was incubated for 2 hours in serum free R10F. This was followed by a 24 hour incubation in the same medium with 10% FCS before reduction of FCS to 2% for a further 24 hours. RSV was harvested by mechanical removal of cells and supernatant, sonication, and snap freezing of aliquots at −80° C. Infectivity was determined by infection of HEp-2 cell monolayers for 2 hours at 37° C. with 50 μl virus stock diluted in RPMI, prior to overlaying with 150 μl R10F. After 48 hours the monolayer was washed with PBS 1% BSA before fixing with 100 μl methanol 0.6% H₂O₂ for 20 minutes. Cells were stained for anti-RSV-HRP (Biogenesis, Poole, Dorset) diluted in PBS/BSA. Cells were washed twice and plaques visualised by 30 minutes incubation with 3-amino-ethylcarbazole (AEC) substrate (0.06 mg/ml AEC, hydrogen peroxide, 6 mM citric acid, 52.6 mM sodium phosphate) before being counted under light microscopy.

### 2.4 Infection of RSV.

BALB/c mice were anaesthetised and infected intranasally with 50 μl 1.4×10⁶ pfu/ml RSV on day 0. One group of mice also received 250 μg A9 antibody intra-peritoneally on days 1 and 4. Weight and appearance of mice was monitored daily. On days 3 or 7 mice were sacrificed by injection of 3 mg pentobarbitone and exsanguination through the femoral artery. Lung, NALT, mediastinal lymph node and spleen were removed; bronchioalveolar lavage was performed by inflating the lungs six times with 1 ml of 1 mM EDTA in EMEM.

### 2.6 Cell Recovery.

Blood removed from the femoral artery was centrifuged at 8000 rpm for 8 minutes and the serum removed and stored at −70° C. BAL washes were centrifuged and the supernatant stored at −20° C.; the pellet was resuspended in R10F, cell counts performed using trypan blue to exclude dead cells, and 2×10⁵ cells used per stain for flow cytometry. Lung tissue, lymph nodes, spleen and NALT were made into a single cell suspension by passing through a 100 μM sieve. This was spun at 1200 rpm for 5 minutes before red blood cells were lysed in 0.15M ammonium chloride, 1M potassium carbonate and 0.001 mM EDTA, and the cells were washed in R10F. Cell pellets were resuspended in R10F and 2×10⁵ used per stain.

### 2.7 Flow Cytometry.

All antibodies were purchased from BD Pharmingen (Heidelberg, Germany) and diluted in PBS/1% BSA/0.05% sodium azide (PBA). Cells were stained for thirty minutes at 4° C., washed in PBA, and centrifuged at 1200 rpm for 5 minutes. When necessary a secondary streptavidin staining step was performed for 20 minutes at 4° C. Cells were washed again and fixed for 20 minutes at room temperature with 2% formaldehyde/PBS. Cells were then washed with and resuspended in PBA, data acquired and 30 000 events analysed with CellQuest Pro software (BD Biosciences, Belgium). To detect intracellular cytokines, cells were incubated with 50 ng/ml PMA, 500 ng/ml ionomycin and 10 mg/ml brefeldin A for 4 hours at 37° C. Cells were surface stained and fixed as before. After permeabilization with PBA containing 1% saponin for 10 minutes, cells were stained with anti-IFN-γ, TNF-α or IL-10. Cells were then washed in PBA/saponin and in PBA alone and run as before. The foxp3 staining was performed with a Foxp3 staining kit (ebioscience) by staining surface molecules as above, then washing cells and incubating overnight with fix and permeabilization solution. Cells were washed again with permeabilization solution and anti-foxp3 PE-conjugated antibody added, followed by incubation for 30 minutes at 4° C. Cells were washed again, resuspended with PBA and run through the flow cytometer within an hour.

### 2.8 Cytokine ELISAs.

Cytokine secretion was quantified with OptEIA kits (BD Pharmingen). Microtitre plates (Nunc, Denmark) were coated with 100 μl capture antibody overnight at 4° C. then blocked with PBS 10% FCS for one hour at room temperature. Samples and standards were diluted in PBS/FCS and loaded before the plates were incubated for 2 hours at room temperature. Bound TNF, IL-10 or IL-12 was detected with a biotinylated antibody and avidin—HRP followed by tetramethylbenzidine and hydrogen peroxidase. Optical densities were read at 450 nm and concentrations calculated from a standard curve.

### 2.9 RSV Specific Antibody ELISAs.

ELISA antigen was prepared by infecting HEp-2 cells with RSV at 1 pfu/cell. The infected cells were harvested, centrifuged at 400 g, resuspended in 3 ml distilled water and sonicated for 2 minutes. 50 μl aliquots were stored at −20° C. Microtitre plates were coated overnight with 100 μl of a 1:200 dilution of the sonicated RSV in distilled water. Wells were blocked with 2% rabbit serum for 2 hours and samples added before a further hour's incubation at room temperature. Bound antibody was detected by incubating with O-phenylenediamine (OPD, Sigma) in the dark for 20 minutes. The reaction was stopped with 50 μl 2M sulphuric acid and plates were read at 490 nm.

### 2.10 RSV—Specific Plaque Assay.

RSV—infected lungs were homogenised, doubly diluted in RPMI and plated out on Hep—2 cells. After 24 hours the cells were overlaid with R10F. 24 hours later the monolayer was washed in PBS 1% BSA before fixing with 100 μl methanol and 0.6% H₂O₂ for 20 minutes. Cells were stained for anti-RSV-HRP and washed twice before plaques were visualised by 30 minute incubation with 3 amino-ethylcarbazole substrate (0.06 mg/ml AEC, hydrogen peroxide, 6 mM citric acid, 52.6 Mm sodium phosphate). Plaques were counted under light microscopy.

### 2.11 NO Assay.

Greiss kits were used to quantify the concentration of nitrite in cell culture supernatants. Samples and standards were treated with 1% sulfanilamide for 10 minutes before addition of 0.1% napthylethylenediamine in 2.5% H₃PO₄, which produces a magenta colour in the presence of nitrite. Optical densities were read at 550 nm and concentrations calculated from a standard curve.

### 2.12 CFSE Staining.

Following purification, CD4 T cells were labelled with the intracellular fluorescent dye 5-carboxyfluorescein diacetate succinimidyl ester (CFSE) to analyse cell division. Cells were resuspended in PBS at 5×10⁷/ml and CFSE added quickly to a final concentration of 10 μM. This was left for ten minutes at room temperature and washed twice in R10F to block the reaction. Cells were then resuspended in R10F for plating.

### 2.13 Detection of Endocytosis.

RAW macrophages were plated as above in the presence or absence of IFN-γ and OX40:1 g for 4 hours. Wells were then washed twice in PBS and cells removed by scraping. Samples were then incubated at 37° C. in the dark with 1 mg/ml FITC-conjugated dextran for 2 hours. Samples were washed again, spun at 1200 rpm for 5 minutes and resuspended in 200 μl PBA before being analysed on the flow cytometer within three hours.

### 2.14 Statistics.

Unless stated otherwise, all experiments were performed at least twice, analysing 5 mice per time point for in vivo experiments and three samples per time point for in vitro assays. Statistical significance was evaluated using the student t test, 2 tailed, assuming unequal variance.

### 3—Discussion of the Results of Example 1—Blocking OX40 During Respiratory Syncytial Virus Infection

### 3.1 Introduction.

It has previously been shown that inhibition of OX40 using a soluble OX40: Tg fusion protein ameliorates the symptoms of influenza virus infection without compromising viral clearance. RSV infection also induces a large influx of CD4+ and CD8+ T cells, neutrophils and macrophages into the lung and airways leading to the occlusion of the alveolar spaces and reduced oxygen transfer. We hypothesis that OX40 inhibition will also reduce this cellular infiltrate, ameliorating the severity of disease, without compromising viral clearance. In the following study we use a pegylated anti-OX40 antibody (A9) to block the interaction between OX40 on the T cells and OX40L on APCs. The main benefits of using A9 versus fusion protein include reduced production costs and prolonged half-life in vivo.

### 3.2 Results.

### 3.2.1 RSV Infection Induces Pulmonary Inflammation and OX40 Expression in the Lung and Mediastinal Lymph Nodes

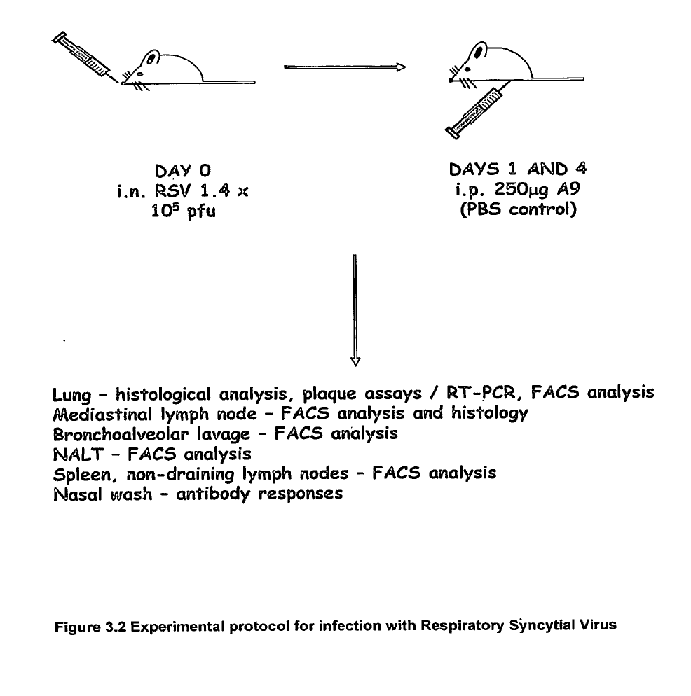

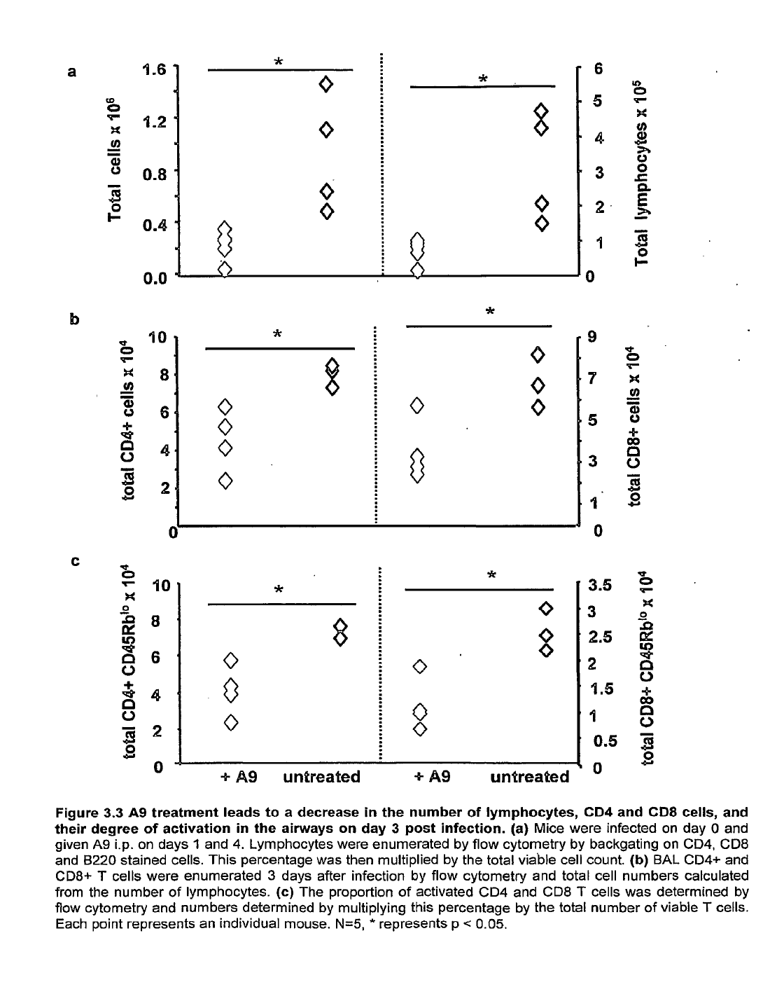

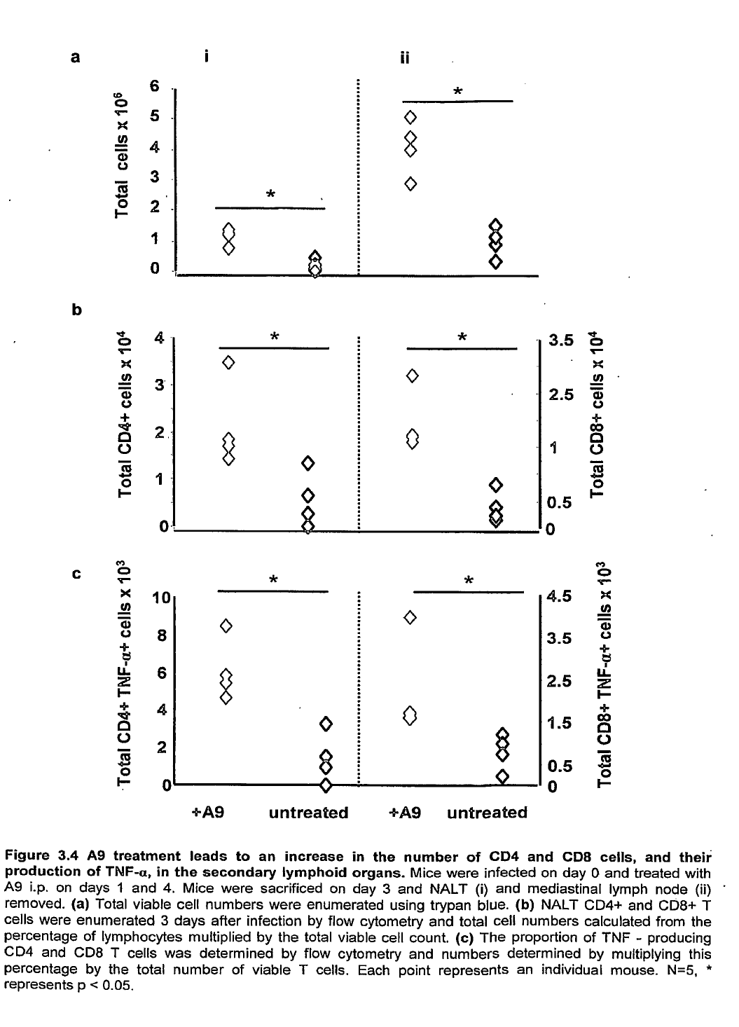

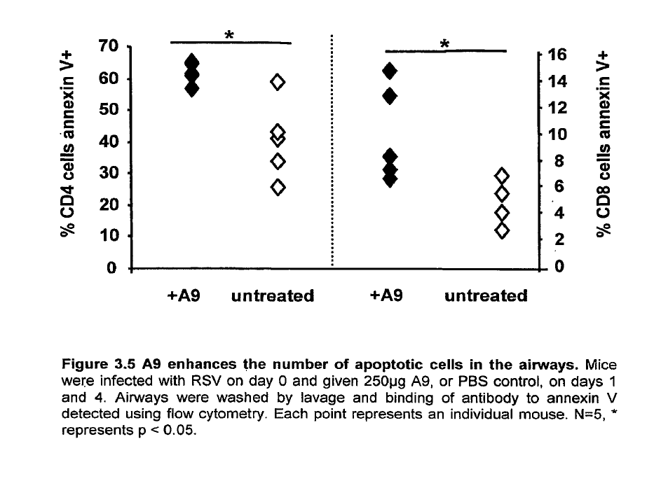

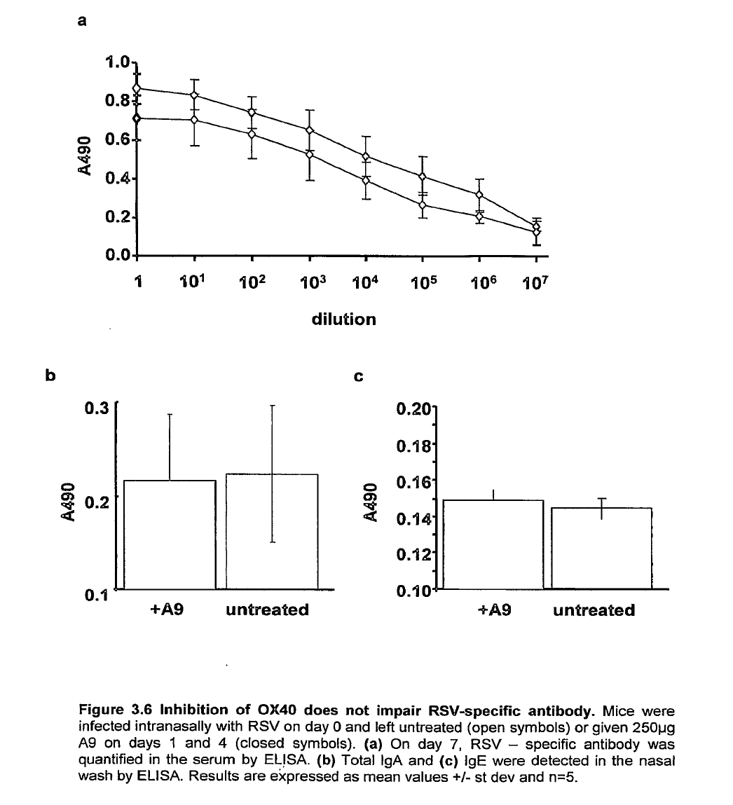

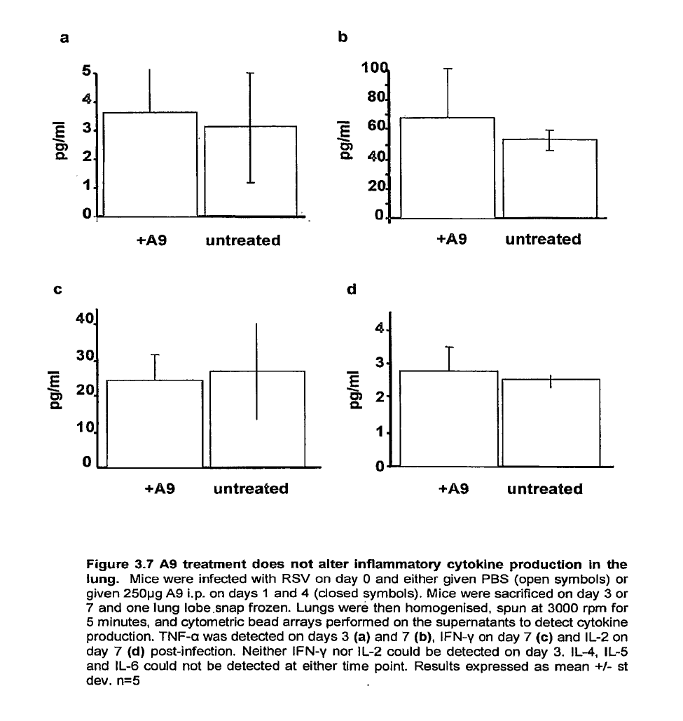

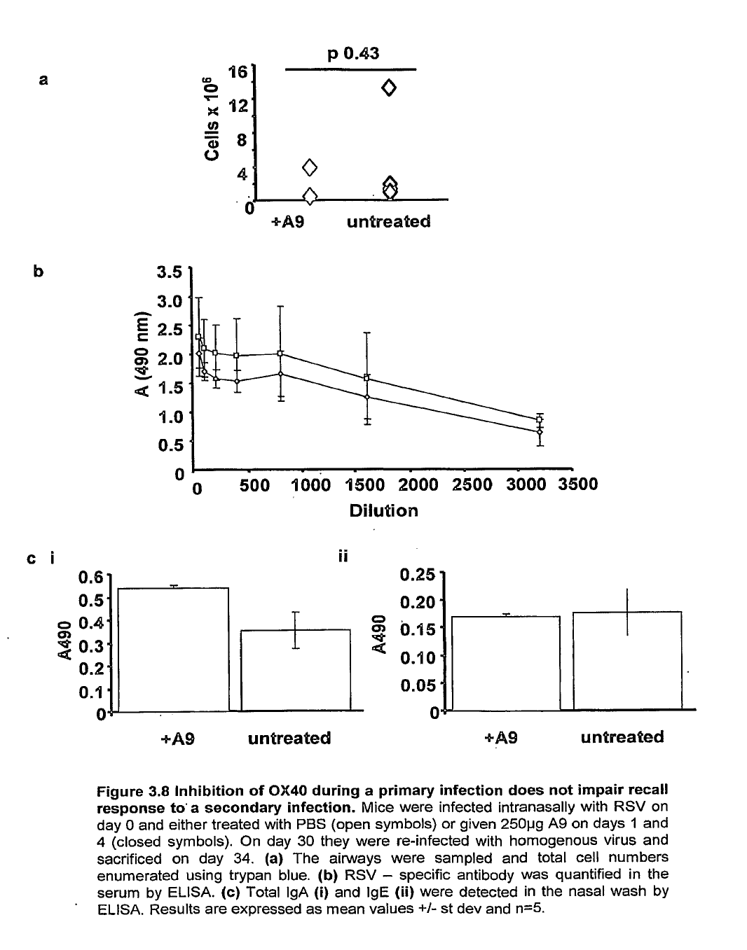

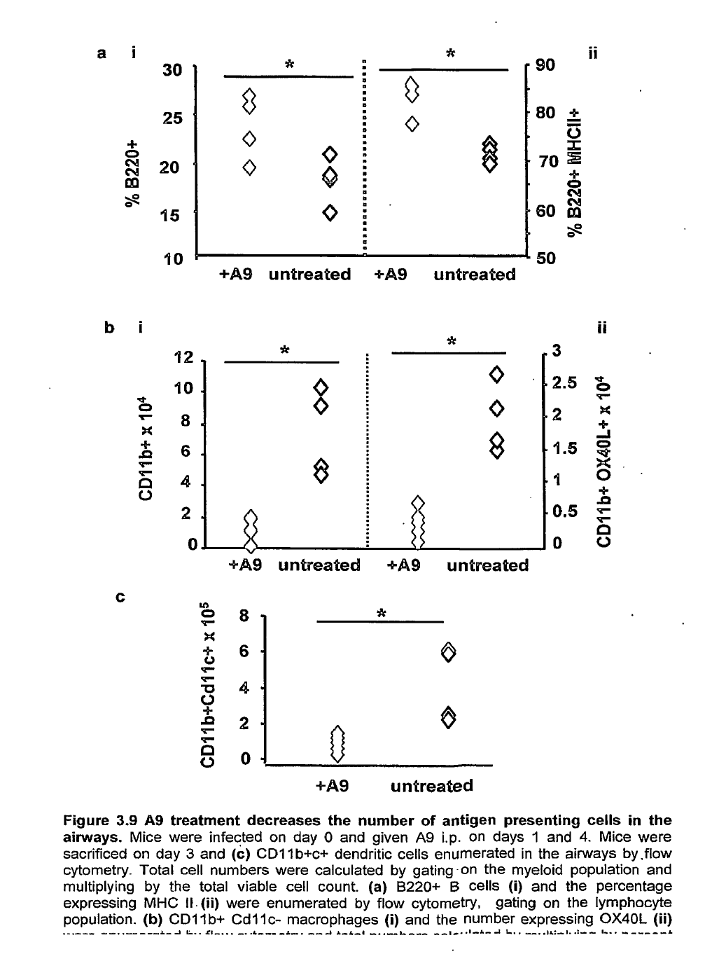

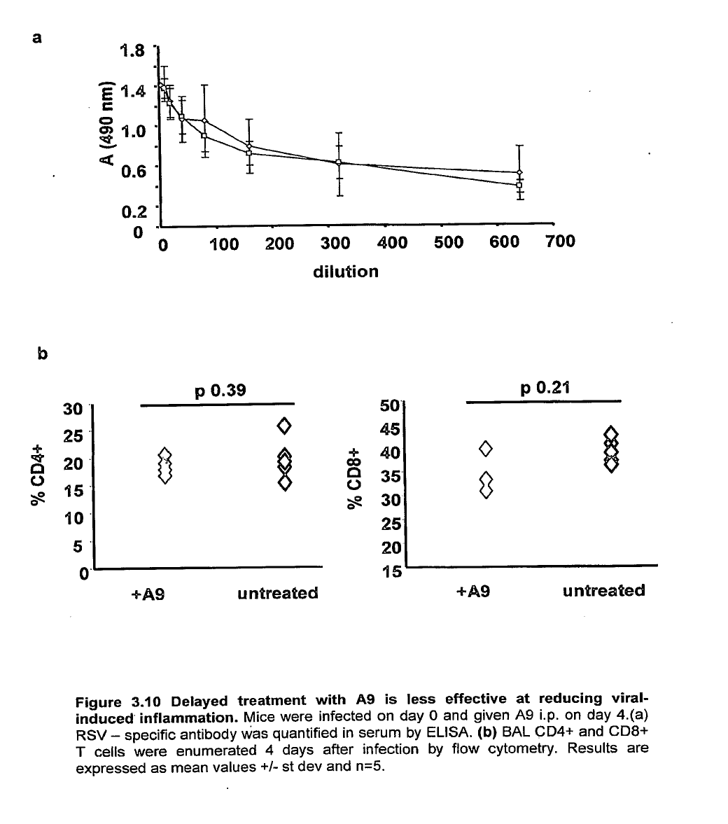

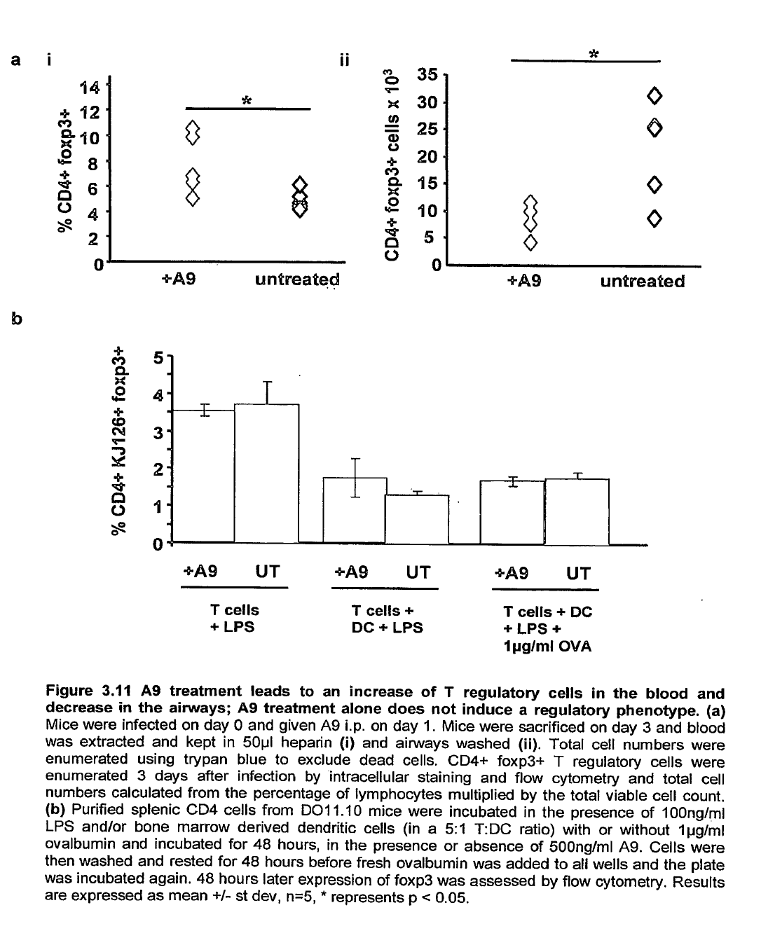

Intranasal infection of BALB/c mice with RSV results in infiltration of lymphocytes into the lungs and airways within three days. The percentage of cells expressing OX40 on days 3 and 7 post-infection was determined using flow cytometry. OX40 was expressed on both CD4 and CD8 cells in the lung, airways, and the mediastinal lymph node. Total numbers of OX40-positive cells were greatly enhanced upon infection. (See FIG. 3.1 which illustrates that RSV infection induces cellular infiltrate into the lungs and OX40 expression in the lung, BAL, and mediastinal lymph node. BALB/c mice were infected with RSV or PBS control on day 0 and sacrificed on day 3. Lavage was performed and then lungs and mediastinal lymph nodes removed, homogenized, and total viable cell counts determined using trypan blue to exclude dead cells. The proportion of OX40 expressing CD4+ and CD8+ T cells was determined by flow cytometry and numbers calculated by multiplying the percentages by the number of CD4 and CD8 cells, and the total viable cell count. OX40 expression was visualized in (a) the lung (b) the MLN and (c) the bronchioalveolar lavage. (d) is a table depicting the mean and standard deviation of 4 mice, representative of 2 experiments. N=4, * represents p<0.005 compared to naïve mice.)

### 3.2.2 OX40 Inhibition Reduces Viral-Induced Inflammation.

To determine whether disruption of OX40—OX40L leads to suppression of RSV—induced immunopathology, 250 μg of a pegylated antibody that binds OX40L on APCs and prevents the association with OX40 on T cells was administered on days 1 and 4 of an RSV infection (See FIG. 3.2 which illustrates an experimental protocol for infection with Respiratory Syncytial Virus). We delayed treatment until day 1 as OX40 expression is not detected in naïve mice.

OX40 inhibition by A9 led to a significant decrease in cellular infiltrate into the lungs and airways which was mostly accounted for by a reduction in CD4+ and CD8+ T cells. Furthermore, fewer were activated, as assessed by CD45Rb^{lo} expression (See FIG. 3.3 which shows that A9 treatment leads to a decrease in the number of lymphocytes, CD4 and CD8 cells, and their degree of activation in the airways on day 3 post infection (a) Mice were infected on day 0 and given A9 i.p. on days 1 and 4. Lymphocytes were enumerated by flow cytometry by backgating on CD4, CD8 and B220 stained cells. This percentage was then multiplied by the total viable cell count. (b) BAL CD4+ and CD8+ T cells were enumerated 3 days after infection by flow cytometry and total cell numbers calculated from the number of lymphocytes. (c) The proportion of activated CD4 and CD8 T cells was determined by flow cytometry and numbers determined by multiplying this percentage by the total number of viable T cells. Each point represents an individual mouse. N=5, * represents p<0.05).

### 3.2.3 A9 Causes The Retention Of Activated Cells In The Secondary Lymphoid Organs.

The reduction of cells in the airways may reflect retention in other sites. The mediastinal lymph nodes (MLN) and Nasal Associated Lymphoid Tissue (NALT) are sites of T cell priming in the respiratory tract. It is therefore possible that reduced priming by A9 treatment prevents cell migration into the airways.

To support this hypothesis, inhibition of OX40 by A9 increased cellularity in the NALT and MLN (FIG. 3.4a). (See FIG. 3.4 which shows that A9 treatment leads to an increase in the number of CD4 and CD8 cells, and their production of TNF-α, in the secondary lymphoid organs. Mice were infected on day 0 and treated with A9 i.p. on days 1 and 4. Mice were sacrificed on day 3 and NALT (i) and mediastinal lymph node (ii) removed. (a) Total viable cell numbers were enumerated using trypan blue. (b) NALT CD4+ and CD8+ T cells were enumerated 3 days after infection by flow cytometry and total cell numbers calculated from the percentage of lymphocytes multiplied by the total viable cell count. (c) The proportion of TNF-producing CD4 and CD8 T cells was determined by flow cytometry and numbers determined by multiplying this percentage by the total number of viable T cells. Each point represents an individual mouse. N=5, * represents p<0.005.) Both CD4+ and CD8+ T cells were increased (FIG. 3.4b). Of those retained, a significantly larger number were activated (data not shown). Furthermore, in the NALT, the number of T cells producing TNF was significantly increased (FIG. 3.4c).

### 3.2.4 OX40 Inhibition Reduces T Cell Numbers By Enhancement Of Apoptosis.

Reduced cell numbers in the airways may also reflect enhanced apoptosis. The level of apoptosis in the lung cell compartments was assessed through flow cytometric analysis of annexin V, which is exposed on a cell when the membrane turns over early in the apoptotic process. Indeed, apoptosis of CD4 and CD8 T cells was increased significantly by A9 treatment in the airways (FIG. 3.5) and in the lung (data not shown). (See FIG. 3.5 which shows A9 enhances the number of apoptotic cells in the airways. Mice were infected with RSV on day 0 and given 250 μg A9, or PBS control, on days 1 and 4. Airways were washed by lavage and binding of antibody to annexin V detected using flow cytometry. Each point represents and individual mouse. N=5, * represents p<0.05.)

3.2.5 OX40 Inhibition does not Reduce Antibody Levels or Control of Viral Replication.

To determine whether the reduction in the number of T cells entering the lung prevented viral containment, plaque assays were performed on snap-frozen lung from mice sacrificed on days 3 and 7 after infection. By day 7 all virus had been cleared from the lung and on day 3 there was no significant difference in the number of plaques present in the untreated and the A9-treated mice. Treatment with A9 did not therefore alter the clearance of the virus from the lung.

Reduced T cell activation may also affect T-dependent antibody production. Total RSV-specific antibody in serum was therefore determined by ELISA. (See FIG. 3.6 which shows inhibition of OX40 does not impair RSV-specific antibody. Mice were infected intranasally with RSV on day 0 and left untreated (open symbols) or given 250 μg A9 on days 1 and 4 (closed symbols). (a) On day 7, RSV-specific antibody was quantified in the serum by ELISA. (b) Total IgA and (c) IgE were detected in the nasal wash by ELISA. Results are expressed as mean values +/−st dev and n=5.) No significant difference in the serum antibody titre on day 3 or 7 between the treated and untreated groups was observed (FIG. 3.6a). IgA and IgE are present at almost undetectable levels in serum but concentrated at mucosal sites. The production of IgA and IgE was therefore assessed by ELISA on nasal wash samples. Blockade of OX40 did not alter the production of these antibodies on days 3 or 7 (FIGS. 3.6b and c).

3.2.6 OX40 Inhibition does not Affect Cytokine Production in the Lung.

Bystander damage to lung tissue can occur due to the production of inflammatory cytokines by T cells and macrophages. We therefore determined whether blockade of OX40 by A9 altered the production of these cytokines, using cytometric bead array technology. However, we did not observe any difference in IL-4, IL-5 or IL-6 by A9 treatment. IFN-γ was only detected at day 7 whereas TNF was abundant at days 3 and 7. Again, there was no effect of A9 treatment (See FIG. 3.7 which shows A9 treatment does not alter inflammatory cytokine production in the lung. Mice were infected with RSV on day 0 and either given PBS (open symbols) or given 250 μg A9 i.p. on days 1 and 4 (closed symbols). Mice were sacrificed on day 3 or 7 and one lung lobe snap frozen. Lungs were then homogenized, spun at 3000 rpm for 5 minutes, and cytometric bead arrays performed on the supernatants to detect cytokine production. TNF-α was detected on days 3 (a) and 7 (b). IFN-γ on day 7 (c) and IL-2 on day 7 (d) post-infection. Neither IFN-γ nor IL-2 could be detected on day 3. IL-4, IL-5, and IL-6 could not be detected at either time point. Results expressed as mean+/−st dev. N=5.)

### 3.2.7 A9 Does not Impair Recall Responses to RSV.

To examine whether reduced cellularity by A9 treatment during a primary infection compromised the ability to clear a second infection, mice were re-challenged four weeks after the original infection and were then sacrificed 4 days later. (See FIG. 3.8 which shows inhibition of OX40 during a primary infection does not impair recall response to a secondary infection. Mice were infected intranasally with RSV on day 0 and either given PBS (open symbols) or given 250 μg A9 i.p. on days 1 and 4 (closed symbols). On day 30 they were re-infected with homogeneous virus and sacrificed on day 34. (a) The airways were sampled and total cell numbers enumerated using trypan blue. (b)) RSV-specific antibody was quantified in the serum by ELISA. (c) Total IgA (i) and IgE (ii) were detected in the nasal wash by ELISA. Results are expressed as mean values +/−st dev and n=5.) Cell recruitment to the airways was still lower in the A9 treated group (FIG. 3.8a). However, the number of cells in the lungs, MLN and NALT were similar between the treated and the control treated groups (data not shown). Furthermore, plaque assays indicated that all virus had been cleared from the lung in both groups (data not shown). Total RSV—specific antibody, and production of IgA and IgE, was also not affected by A9 treatment (FIG. 3.8b and c).

### 3.2.8 Blockade of OX40 Reduces Antigen Presenting Cell Numbers in the Airways.

Previous work in the laboratory focussed on using the OX40: Ig fusion protein to block T cell activation. However, this also delivers a positive signal to the APC bearing OX40L. A9, in contrast, does not. It was therefore hypothesised that use of A9 would also lead to a reduction in the number of APCs present in the airways during infection. (See FIG. 3.9 which shows A9 treatment decreases the number of antigen presenting cells in the airways. Mice were infected on day 0 and given A9 i.p. on days 1 and 4. Mice were sacrificed on day 3 and (c) CD11b+c+dendritic cells enumerated in the airways by flow cytometry. Total cell numbers were calculated by gating on the myeloid population and multiplying by the total viable cell count

(a) B220+B cells (i) and the percentage expressing MHC II (ii) were enumerated by flow cytometry, gating on the lymphocyte population. (b) CD11b+Cd11c-macrophages (i) and the number expressing OX40L (ii) were enumerated by flow cytometry and total numbers calculated by multiplying by percent in the myeloid gate and total viable cell counts. Each point represents an individual mouse, n=5. *=p<0.05.) To investigate this, lungs and mediastinal lymph nodes were removed, homogenised and the numbers of DCS (CD11c+) macrophages (Cd11b+) and B cells (B220+) were determined. The intensity of MHC class 11 expression was used to study activation of these cells, and compared to the expression of OX40L.

The percentage of B220+ cells was actually increased in both the NALT and the lung A9 treatment on day 7 (FIG. 3.9a). However, the actual numbers were unaltered. Those B cells present in the airways, however, were more activated by A9 treatment.

The number of macrophages present in the lung and airways of A9 treated mice were also lower day 3, but not day 7, post-infection (FIG. 3.9b). The number of macrophages that stained positive for OX40L was also significantly decreased. Furthermore, CD11c+ dendritic cells were also significantly decreased in the airways and the lungs on day 3 post infection (FIG. 3.9c).

### 3.2.9 Delayed Treatment of A9 is Less Effective at Reducing Viral-Induced Inflammation.

In a clinical setting, giving A9 one day after infection may not be realistic, since it is before the onset of clinical symptoms of disease (on day 3). (See FIG. 3.10 which shows delayed treatment with A9 is less effective at reducing viral-induced inflammation.) To evaluate whether a late administration of A9 is also effective at reducing immunopathology, mice were infected with RSV on day 0 and 250 μg A9 given on day 4 only. Mice were sacrificed on day 7 and lungs, airway and draining lymph nodes analysed. (a) RSV-specific antibody was quantified in serum by ELISA. (b) BAL CD4+ and CD8+ T cells were enumerated 4 days after infection by flow cytometry. Results are expressed as mean values +/−st dev and n=5.) RSV-specific antibody in serum was unaltered by delayed treatment (FIG. 3.10a). Plaque assays also determined that there was no difference in viral titres (data not shown). T cells were also reduced but this did not reach significance. Future experiments may determine whether either treatment at day 3 or 3 post-infection or a higher dose at day 4 is efficacious (FIG. 3.10b).

3.2.10 Blockade of OX40 does not Affect Development of Memory Cells.

Ligation of OX40 on the T cell induces proliferation, up-regulates the anti-apoptotic proteins of the Bcl family, and is thought to have a role in seeding the memory T cell pool. It was a concern, therefore, that blocking the interaction of OX40 with its ligand would prevent the development of memory T cells. To investigate this, T cell subsets were analysed in mice treated with A9 during a primary infection and rechallenged with homogenous virus 30 days later in the spleen, lymph nodes, lungs and airways. T cells were determined to be central memory cells (CD44^{hi} CD62^{lo}), effector memory cells (CD44^{hi} CD62L^{hi}), or naïve cells (CD44^{lo} CD62L^{hi}), according to the criteria of Lazavecchia. No differences in the numbers of any of these cell populations by A9 treatment compared to the untreated group was observed (data not shown). CCR7 is used to distinguish naïve and central memory cells (positive) from effector memory (negative). There was no difference seen in the expression of CCR7 between the treated and untreated groups (data not shown).

### 3.2.11 Blockade of OX40 Induces Expansion of Regulatory T Cell Populations in the Blood but Reduces Them in the Airways.

Recently, much work has concentrated on the role of regulatory T cells in infection. It was therefore interesting to examine whether the reduced infiltrate into the lungs and airways was due to increased numbers of regulatory T cells. The precise phenotype of these cells is controversial, however we decided to enumerate them using intracellular foxp3 staining.

There was a significant increase in foxp3+ cells in the peripheral blood on day 3 post-infection in the A9, compared to the control, treated group. In the airways however they were significantly reduced. (See FIG. 3.11 which shows A9 treatment leads to an increase of T regulatory cells in the blood and decrease in the airways; A9 treatment alone does not induce a regulatory phenotype. (a) Mice are infected on day 0 and given A9 i.p. on day 1. Mice were sacrificed on day 3 and blood was extracted and kept in 50 μl heparin (i) and airways washed (ii). Total cell numbers were enumerated using trypan blue to exclude dead cells. CD4+ foxp3+ T regulatory cells were enumerated 3 days after infection by intracellular staining and flow cytometry and total viable cell count numbers calculated from the percentage of lymphocytes multiplied by the total viable cell count. (b) Purified splenic CD4 cells from DO11.10 mice were incubated in the presence of 100 ng/ml LPS and/or bone marrow derived dendritic cells (in a 5:1 T:DC ratio) with or without 1 μg/ml ovalbumin and incubated for 48 hours, in the presence or absence of 500 ng/ml A9. Cells were then washed and rested for 48 hours before fresh ovalbumin was added to all wells and the plate was incubated again. 48 hours later expression of foxp3 was assessed by flow cytometry. Results are expressed as mean+/−st dev. N=5,)

Regulatory T cells express OX40 and so it was important to determine whether the alteration in their numbers is due to a blockade in their migration from the blood, as has been seen with other cell types, or whether inhibition of OX40 signalling forces peripheral CD4+ cells into a regulatory phenotype. To address this, CD4+ T cells were purified from DO11.10 mice and incubated with 1 μg/ml ovalbumin and bone marrow—derived dendritic cells in the presence or absence of A9. After 48 hours, cells were washed and rested in fresh medium for a further 48 hours, before fresh ovalbumin was added and the activation and phenotype of the cells assessed. There was no difference in the proportion of cells staining positive for foxp3, indicating that A9 alone does not induce a regulatory phenotype (FIG. 3.11b).

### Example 2

### Influenza Virus

The experiments of Example 1 were repeated using Influenza virus instead of RSV.

### 4—Materials and Methods

The materials and methods used with respect to influenza virus were the same as for RSV with the exception that Influenza X-31 (obtained from the National Institute of Medical Research, Mill Hill, London) was a administered intranasally at a dosage of 50 μL 58 HA units of Influenza X-31. In all other respects, the materials and methods set forth in sections 2.1 through 2.14 were used.

### 5. Discussion of the Results of Example 2—OX40 During Influenza Infection

5.1 Influenza Infection of Balb/C Mice Elicits an Acute Weight Loss that Peaks at Day 6-7 After Infection.

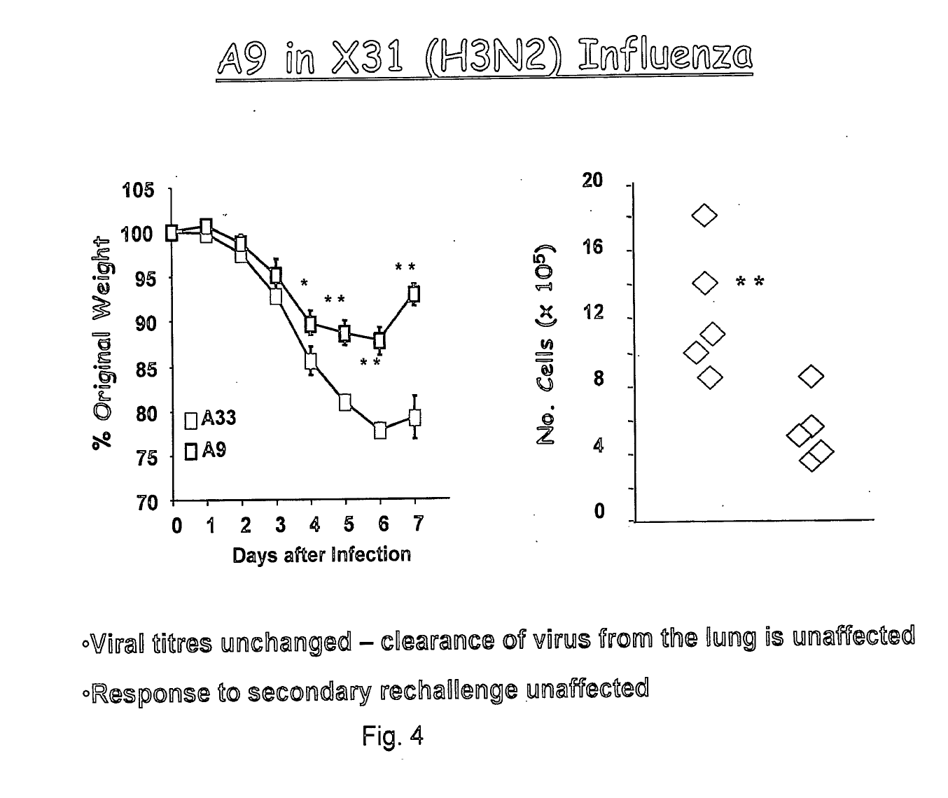

As may be seen with respect to FIG. 4, influenza infection of BALB/c mice elicits an acute weight loss that peaks at day 6-7 after infection. The inflammatory infiltrate into the airways is also maximal at this time point implying a strong correlation between illness severity and the exuberance of the host's immune response. A significant extent of the observed illness and pathology evoked by influenza infection of the lung is attributable to the over-exuberance of the host's immune response. T cells are critical to viral clearance but are also a significant component of the observed pathology, causing occlusion of airways and producing inflammatory mediators that cause the observed cachexia and fever. The dependence of illness on T cell accumulation can easily be demonstrated by inhibiting the late co-stimulatory signal delivered through OX40. BALB/c mice were infected intranasally with 50HA influenza on day 0 and administered a pegylated anti-OX40 blocking antibody (A9) or control antibody (A33) intraperitoneally on days 0 and 3 after infection. Administration of A9 significantly reduced weight loss following influenza infection of BALB/c mice compared to mice treated with control antibody A33 (FIG. 4, left). Those mice treated with A9 also exhibited significantly reduced number of cells in their airways at day 7 post infection (FIG. 4 right). Flow cytometric analysis confirmed that blockade of the OX40 signal to the T cell reduces the number of CD4⁺ and CD8⁺ T cells in the airways, and their production of intracellular IFN-γ and TNF cytokines, 7 days after infection. Despite the significantly reduced number of T cells in the airways of the A9 treated mice, these mice were still able to clear the virus from the lungs, at a comparable rate to control treated mice. Furthermore, the memory response in the A9 treated mice was unaltered and these mice were able to successfully and rapidly clear a secondary exposure to influenza.

### 5.2 Conventional Strategies to Combat Influenza Infection Rely Upon Vaccination and Administration of Antiviral Drugs.

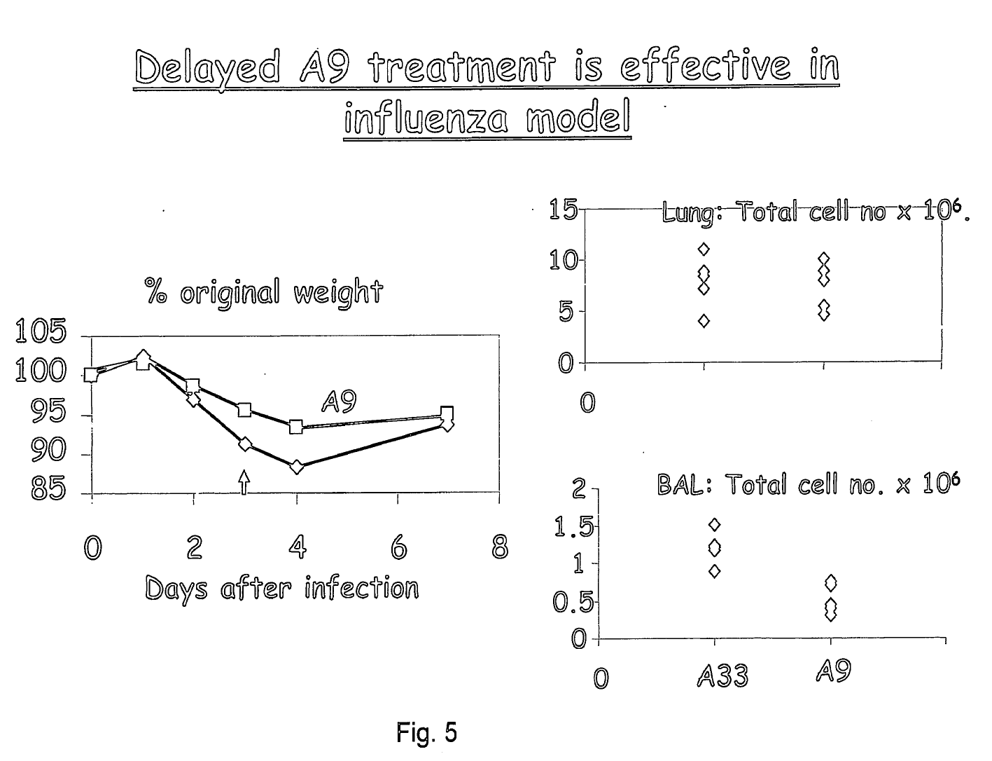

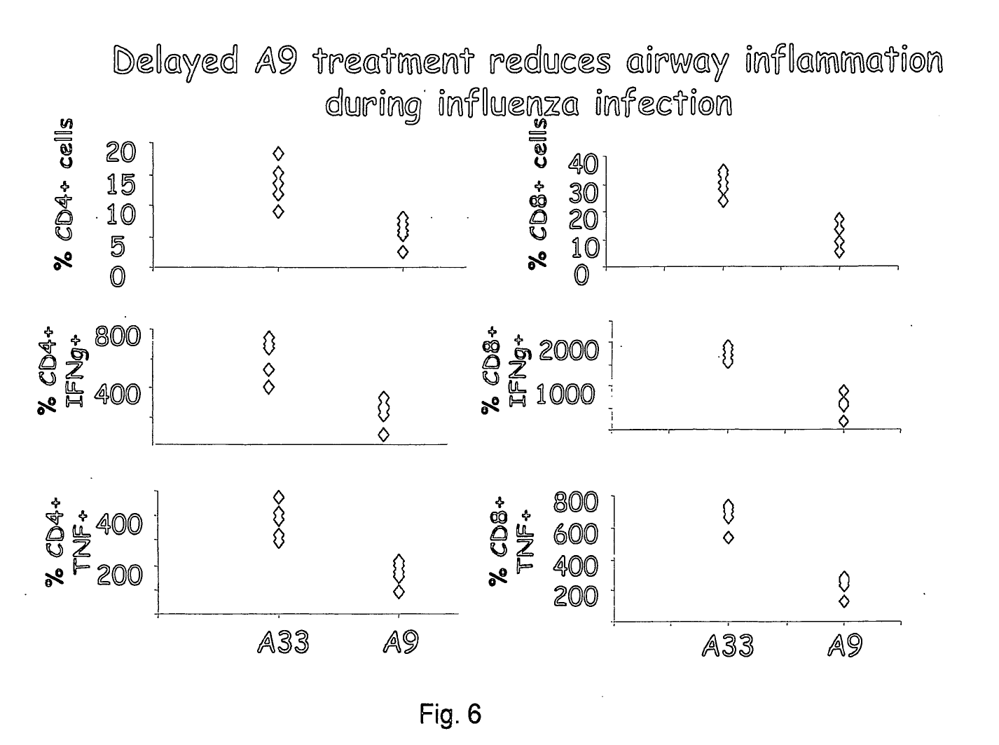

As may be seen with respect to FIG. 5, conventional strategies to combat influenza infection rely upon vaccination and administration of antiviral drugs. Vaccination strategies are hindered by the antigenic variation of the pathogen whereas anti-microbial agents are sometimes limited by efficacy and increasing incidences of drug resistance. Furthermore, in infections such an influenza clinical signs of disease are only really apparent when viral titres have subsided 1.5 rendering anti-virals ineffective. One advantage of A9 treatment is that it can be utilised therapeutically (after infection) when symptoms start to arise. Mice administered A9 at day 3 after influenza infection still showed reduced weight loss relative to control treated mice and a reduction in the number of cells in their airways.

### 5.3 The Delayed Treatment of A9 Resulted in a Significant Reduction in the Number of CD4+ and CD8+ T Cells in the Airways Relative to Control Treated Mice.

As may be seen with respect to Slide 6, the delayed treatment of A9 resulted in a significant reduction in the number of CD4+ and CD8+ T cells in the airways relative to control treated mice. (See, Slide 3) The levels of pro-inflammatory cytokines, IFN-gamma and TNF, released by T cells are implicated in much of the observed illness and pathology. Importantly, A9 treatment reduced the numbers of CD4+ and CD8+ T cells producing both IFN-gamma and TNF relative to control treated (A33) mice.

The present invention is susceptible to modifications and variations as will be apparent to those skilled in the art in light of the disclosure herein, and the present disclosure extends to combinations and subcombinations of the features mentioned or described herein.

1. A composition comprising a molecular blockade agent to a costimulatory molecule said costimulatory molecule being
a. absent in naïve or resting T-lymphocytes;
b. inducible;
c. expressed; and
d. prominent at the height of an immunopathological response.

2. A composition in accordance with claim 1 wherein said costimulatory molecule is a receptor or a ligand.

3. A composition in accordance with claim 1 wherein said immunopathological response is a disease response or a condition response

4. A composition as recited in claim 1 wherein said costimulatory molecule comprises
a. OX40,
b. 4-1BB,
c. CD27,
d. CD30,
e. HVEM,
f. GITR,
g. ICOS,
h. PD1, or
i. CTLA4
g. a derivative of the foregoing in which activity is conserved,
h. a variant of the foregoing in which activity is conserved, or
i. a combination of two or more of the foregoing.

5. A composition as recited in claim 1 wherein said costimulatory molecule comprises
a. OX40 ligand,
b. 4-1BB ligand,
c. CD70,
d. CD30 ligand,
e. LIGHT,
f. GITR ligand,
g. a derivative of the foregoing in which activity is conserved,
h. a variant of the foregoing in which activity is conserved, or
i. a combination of two or more of the foregoing.

6. A composition as recited in claim 1 wherein said immunopathological response is a response to an infective agent or a traumatic agent.

7. A composition as recited in claim 1 wherein said immunopathological response is a response to
a. a peptide,
b. a polypeptide,
c. a nucleotide,
d. an antigen, or
e. a combination of two or more of the foregoing.

8. A composition as recited in claim 6 wherein said infective agent comprises
a. a multicellular infective agent,
b. a bacterial infective agent,
c. a fungal infective agent,
d. a viral infective agent,
e. a prion infective agent, or
f. a combination of two or more of the foregoing.

9. A composition as recited in claim 1 wherein said infective agent comprises influenza.

10. A composition as recited in claim 1 wherein said infective agent comprises pandemic influenza.

11. A composition as recited in claim 1 wherein said infective agent comprises avian influenza A (H5N1).

12. A composition as recited in claim 1 wherein said traumatic agent comprises
a. a biological traumatic agent
b. a chemical traumatic agent
c. a nuclear traumatic agent
d. a mechanical traumatic agent
e. a combination of two or more of the foregoing.

13. A composition comprising a modulating agent for a signal pathway of a costimulatory molecule, said costimulatory molecule being
a. absent in naïve or resting T-lymphocytes;
b. inducible;
c. expressed; and
d. prominent at the height of an immunopathological response

14. A composition as recited in claim 13, wherein said pathway includes one or more of said pathway's extracellular components, transmembrane components, or intracellular components or a combination of two or more of the foregoing.

15. A composition as recited in claim 13, wherein said pathway includes a TRAF 2 component

16. A composition as recited in claim 13, wherein said agent comprises a modified TRAF 2 component

17. A method comprising
a. administering to a subject a molecular blockade agent to a costimulatory molecule said costimulatory molecule being
i. absent in naïve or resting T-lymphocytes;
ii. inducible;
iii. expressed; and
iv. prominent at the height of an immunopathological response by said subject.

18. A method in accordance with claim 17 wherein said administering is prior to the height of said immunopathological response.

19. A method in accordance with claim 17 wherein said administering is contemporaneous with said immunopathological response.

20. A method in accordance with claim 17 wherein said administering is contemporaneous with the height of said immunopathological response.

21. A method in accordance with claim 17 wherein said subject is a mammal.

22. A method in accordance with claim 17 wherein said subject is a human.
